(19) United States
(12) Patent Application Publication
(10) Pub. No.: US 2009/0214651 A1
(43) Pub. Date: Aug. 27, 2009

(54) PRODUCTION OF POLYURETHANE FOAMS

(75) Inventors: Burkhard FUGMANN, Ratingen (DE); Melita Dietze, Erkrath (DE); Michael Mager, Leverkusen (DE); Thorsten Rische, Unna (DE); Sebastian Dorr, Dusseldorf (DE); Thomas Feller, Solingen (DE); Michael Heckes, Krefeld (DE); Jan Schonberger, Solingen (DE)

Correspondence Address: Baker Donelson Bearman, Caldwell & Berkowitz, PC, 555 Eleventh Street, NW, Sixth Floor, Washington, DC, 20004, US

(73) Assignee: BAYER INNOVATION GMBH, Dusseldorf (DE)

(21) Appl. No.: 12/244,371

(22) Filed: Oct. 2, 2008

(30) Foreign Application Priority Data
Oct. 5, 2007 (DE) 10 2007 048 079.4

(51) Int. Cl. C08J 9/30; A61K 9/10; A61K 31/155; A61L 15/22

(52) U.S. Cl. 424/486; 521/65; 514/635; 602/46

(57) ABSTRACT

The invention relates to a process for producing polyurethane foams, by frothing and drying specific polyurethane dispersions.

The invention relates to a process for producing polyurethane foams, by frothing and drying specific polyurethane dispersions.

In the field of wound management, the use of polyurethane foams as a wound contact layer is well known. The polyurethane foams used for this purpose are generally hydrophilic in order that good absorption of wound fluid may be ensured. Hydrophilic polyurethane foams are obtained by reaction of mixtures of diisocyanates and polyols, or NCO-functional polyurethane prepolymers, with water in the presence of certain catalysts and also (foam) additives. Aromatic diisocyanates are typically used, since they are best foamable. Numerous forms of these processes are known, for example described in U.S. Pat. No. 3,978,266, U.S. Pat. No. 3,975,567 and EP-A 0 059 048. However, the aforementioned processes have the disadvantage that they require the use of reactive mixtures, containing diisocyanates or corresponding prepolymers, whose handling is technically inconvenient and costly, since appropriate protective measures are necessary for example.

It is also known to produce foams from polyurethane dispersions by incorporating air in the presence of suitable (foam) additives by vigorous stirring. So-called mechanical polyurethane foams are obtained after drying and curing. In connection with wound contact materials, such foams are described in EP-A 0 235 949 and EP-A 0 246 723, the foam either having a self-adherent polymer added to it, or being applied to a film of a self-adherent polymer. The use of the foams as such, i.e. without self-adherent polymers, is not described. In addition, the examples recited in EP 0 235 949 and EP 0 246 723 mandate the use as crosslinkers of polyaziridines which are now no longer acceptable because of their toxicity. Moreover, crosslinking requires the use of high baking temperatures, reported to be in the range from 100° C. to 170° C. U.S. Pat. No. 4,655,210 describes the use of the aforementioned mechanical foams for wound dressings having a specific construction of backing, foam and skin contact layer.

The polyurethane dispersions described in EP-A 0 235 949, EP-A 0 246 723 and U.S. Pat. No. 4,655,210 are anionically hydrophilicized through incorporation of certain carboxylic acids such as dimethylol carboxylic acids and neutralization of the carboxylic acids with tertiary amines, for example triethylamine. However, the ammonium carboxylates thus formed are decomposable, in particular at higher temperatures, which sets the amines free again. This is an immense disadvantage in relation to the processing of such products and particularly in skin contact. Furthermore, these polyurethane dispersions were produced using the dimethylol carboxylic acids in dissolved form, for example in dimethylformamide or N-methylpyrrolidone, as a result of which the final products have an altogether high VOC content, 10.8 g litre (without water) in the case of the Witcobond™ 290H used.

EP 0 760 743 describes such mechanical foams formed on the basis of latex dispersions, but they do not consist of polyurethanes and have worse mechanical properties.

The present invention therefore has for its object to provide novel wound contact materials which are based on polyurethanes and are obtainable in a very simple manner and without the use of such building block components or additives as are not generally recognized as safe. It is a further prerequisite that these wound contact materials have good mechanical properties, a high uptake capacity for physiological saline and also a high water vapour transmission rate.

It has now been found that such polyurethane-based wound contact materials are obtainable wherein compositions containing specific aqueous polyurethane dispersions are frothed and then physically dried.

The present invention accordingly provides a process for producing wound contact materials which comprises compositions containing anionically hydrophilicized, aqueous polyurethane dispersions (I) being frothed and physically dried without chemical crosslinking.

Crosslinking herein is to be understood as meaning the formation of covalent bonds.

Polyurethane foam wound contact materials for the purposes of the present invention are porous materials, preferably having at least some open-cell content, which consist essentially of polyurethanes and protect wounds against germs and environmental influences like a sterile covering, have a fast and high absorbance of physiological saline or to be more precise wound fluid, have a suitable permeability for moisture to ensure a suitable wound climate, and have sufficient mechanical strength.

Preferably, these dispersions are anionically hydrophilicized by means of sulphonate groups. More preferably, sulphonate groups only are responsible for the anionic hydrophilicization.

Preferably, the specific polyurethane dispersions (I) have a low degree of hydrophilic anionic groups, preferably 0.1 to 15 milliequivalents per 100 g of polyurethane (solid resin).

To achieve good stability to sedimentation, the number average particle size of the specific polyurethane dispersions is preferably less than 750 nm and more preferably less than 500 nm, determined by laser correlation spectroscopy.

The solids contents of the polyurethane dispersions (I) are preferably in the range from 30% to 70% by weight, more preferably in the range from 50% to 70% by weight, even more preferably in the range from 55% to 65% by weight and in particular in the range from 60 to 65% by weight, based on the polyurethane present therein.

The level of unbound organic amines in these polyurethane dispersions is preferably less than 0.5% by weight and more preferably less than 0.2% by weight, based on the entire dispersions.

Such preferred polyurethane dispersions (I) are obtainable by

A) isocyanate-functional prepolymers being produced from
A1) organic polyisocyanates
A2) polymeric polyols having number-average molecular weights in the range from 400 to 8000 g/mol, preferably in the range from 400 to 6000 gμmol and even more preferably in the range from 600 to 30 000 g/mol and OH functionalities in the range from 1.5 to 6, preferably in the range from 1.8 to 3 and more preferably in the range from 1.9 to 2.1, and
A3) optionally hydroxyl-functional compounds having molecular weights in the range from 62 to 399 g/mol and
A4) optionally isocyanate-reactive, anionic or potentially anionic and/or optionally nonionic hydrophilicizing agents
and
   B) its free NCO groups then being wholly or partly reacted
B1) optionally with amino-functional compounds having molecular weights in the range from 32 to 400 g/mol and
B2) with amino-functional, anionic or potentially anionic hydrophilicizing agents
   by chain extension, and the prepolymers being dispersed in water before, during or after step B).

If desired, the prepolymer can be wholly or partly converted into the anionic form by admixing a base, before, during or after dispersion.

To achieve anionic hydrophilicization, A4) and/or B2) shall utilize hydrophilicizing agents that have at least one NCO-reactive group such as amino, hydroxyl or thiol groups and additionally have —COO⁻ or —SO₃⁻ or —PO₃²⁻ as anionic groups or their wholly or partly protonated acid forms as potentially anionic groups.

Preferably, A4) and/or B2) utilize such compounds for anionic or potentially anionic hydrophilicization as have exclusively sulphonic acid or sulphonate groups (—SO₃H or —SO₃M, where M=alkali metal or alkaline earth metal) as anionic or potentially anionic functionality.

Suitable polyisocyanates of component A1) are the well-known aliphatic or cycloaliphatic polyisocyanates having an NCO functionality of not less than 2.

Examples of such suitable polyisocyanates are 1,4-butylene diisocyanate, 1,6-hexa-methylene diisocyanate (HDI), isophorone diisocyanate (IPDI), 2,2,4- and/or 2,4,4-trimethylhexamethylene diisocyanate, the isomeric bis(4,4′-isocyanato-cyclohexyl)methane or their mixtures of any desired isomer content, 1,4-cyclohexylene diisocyanate, 4-isocyanatomethyl-1,8-octane diisocyanate (nonane triisocyanate) and also alkyl 2,6-diisocyanatohexanoates (lysine diisocyanates) having C1-C8-alkyl groups.

As well as the aforementioned polyisocyanates, it is possible to use modified diisocyanates having a functionality≧2 and a uretidione, isocyanurate, urethane, allophanate, biuret, iminooxadiazinedione or oxadiazinetrione structure, and also mixtures thereof pro rata.

Preferably, the polyisocyanates or polyisocyanate mixtures of the aforementioned type have exclusively aliphatically or cycloaliphatically attached isocyanate groups or mixtures thereof and an average NCO functionality in the range from 2 to 4, preferably in the range from 2 to 2.6 and more preferably in the range from 2 to 2.4, for the mixture.

It is particularly preferable for A1) to utilize hexamethylene diisocyanate, isophorone diisocyanate or the isomeric bis(4,4′-isocyanatocyclohexyl)methanes and also mixtures thereof.

A2) utilizes polymeric polyols having a number average molecular weight Mₙ in the range from 400 to 8000 g/mol, preferably from 400 to 6000 g/mol and more preferably from 600 to 3000 g/mol. These preferably have an OH functionality in the range from 1.5 to 6, more preferably in the range from 1.8 to 3 and most preferably in the range from 1.9 to 2.1.

Such polymeric polyols are the well-known polyurethane coating technology polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols. These can be used in A2) individually or in any desired mixtures with one another.

Such polyester polyols are the well-known polycondensates formed from di- and also optionally tri- and tetraols and di- and also optionally tri- and tetracarboxylic acids or hydroxy carboxylic acids or lactones. Instead of the free polycarboxylic acids it is also possible to use the corresponding polycarboxylic anhydrides or corresponding polycarboxylic esters of lower alcohols for preparing the polyesters.

Examples of suitable diols are ethylene glycol, butylene glycol, diethylene glycol, triethylene glycol, polyalkylene glycols such as polyethylene glycol, also 1,2-propanediol, 1,3-propanediol, butanediol(1,3), butanediol(1,4), hexanediol(1,6) and isomers, neopentyl glycol or neopentyl glycol hydroxypivalate, of which hexanediol(1,6) and isomers, butanediol(1,4), neopentyl glycol and neopentyl glycol hydroxypivalate are preferred. Besides these it is also possible to use polyols such as trimethylolpropane, glycerol, erythritol, pentaerythritol, trimethylolbenzene or trishydroxyethyl isocyanurate.

Useful dicarboxylic acids include phthalic acid, isophthalic acid, terephthalic acid, tetra-hydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. The corresponding anhydrides can also be used as a source of an acid.

When the average functionality of the polyol to be esterified is >than 2, monocarboxylic acids, such as benzoic acid and hexanecarboxylic acid can be used as well in addition.

Preferred acids are aliphatic or aromatic acids of the aforementioned kind. Adipic acid, isophthalic acid and phthalic acid are particularly preferred.

Hydroxy carboxylic acids useful as reaction participants in the preparation of a polyester polyol having terminal hydroxyl groups include for example hydroxycaproic acid, hydroxybutyric acid, hydroxydecanoic acid, hydroxystearic acid and the like. Suitable lactones include caprolactone, butyrolactone and homologues. Caprolactone is preferred.

A2) may likewise utilize hydroxyl-containing polycarbonates, preferably polycarbonatediols, having number average molecular weights Mₙ in the range from 400 to 8000 g/mol and preferably in the range from 600 to 3000 g/mol. These are obtainable by reaction of carbonic acid derivatives, such as diphenyl carbonate, dimethyl carbonate or phosgene, with polyols, preferably diols.

Examples of such diols are ethylene glycol, 1,2-propanediol, 1,3-propanediol, 1,3-butane-diol, 1,4-butanediol, 1,6-hexanediol, 1,8-octanediol, neopentyl glycol, 1,4-bishydroxy-methylcyclohexane, 2-methyl-1,3-propanediol, 2,2,4-trimethyl-1,3-pentanediol, dipropylene glycol, polypropylene glycols, dibutylene glycol, polybutylene glycols, bisphenol A and lactone-modified diols of the aforementioned kind.

The diol component preferably contains 40% to 100% by weight of hexanediol, preference being given to 1,6-hexanediol and/or hexanediol derivatives. Such hexanediol derivatives are based on hexanediol and have ester or ether groups as well as terminal OH groups. Such derivatives are obtainable by reaction of hexanediol with excess caprolactone or by etherification of hexanediol with itself to form di- or trihexylene glycol.

In lieu of or in addition to pure polycarbonate diols, polyether-polycarbonate diols can also be used in A2).

Hydroxyl-containing polycarbonates preferably have a linear construction.

A2) may likewise utilize polyether polyols.

Useful polyether polyols include for example the well-known polyurethane chemistry polytetramethylene glycol polyethers as are obtainable by polymerization of tetrahydro-furan by means of cationic ring opening.

Useful polyether polyols likewise include the well-known addition products of styrene oxide, ethylene oxide, propylene oxide, butylene oxides and/or epichlorohydrin onto di- or polyfunctional starter molecules. Polyether polyols based on the at least proportional addition of ethylene oxide onto di- or polyfunctional starter molecules can also be used as component A4) (nonionic hydrophilicizing agents).

Useful starter molecules include all prior art compounds, for example water, butyl diglycol, glycerol, diethylene glycol, trimethylolpropane, propylene glycol, sorbitol, ethylenediamine, triethanolamine, 1,4-butanediol.

A3) may utilize polyols of the specified molecular weight range with up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclo-hexanedimethanol, 1,6-hexanediol, neopentyl glycol, hydroquinone dihydroxyethyl ether, bisphenol A (2,2-bis(4-hydroxyphenyl)propane), hydrogenated bisphenol A, (2,2-bis(4-hydroxycyclohexyl)propane), trimethylolpropane, glycerol, pentaerythritol and also any desired mixtures thereof with one another.

Also suitable are esterdiols of the specified molecular weight range such as α-hydroxy-butyl-ε-hydroxycaproic acid ester, ω-hydroxyhexyl-γ-hydroxybutyric acid ester, β-hydroxyethyl adipate or bis(β-hydroxyethyl) terephthalate.

A3) may further utilize monofunctional isocyanate-reactive hydroxyl-containing compounds. Examples of such monofunctional compounds are ethanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Useful anionically hydrophilicizing compounds for component A4) include salts of mono- and dihydroxy sulphonic acids. Examples of such anionic hydrophilicizing agents are the adduct of sodium bisulphite onto 2-butene-1,4-diol as described in DE-A 2 446 440, pages 5-9, formula I-III.

Useful nonionically hydrophilicizing compounds for component A4) include for example polyoxyalkylene ethers containing at least one hydroxyl, amino or thiol group. Examples are the monohydroxyl-functional polyalkylene oxide polyether alcohols containing on average 5 to 70 and preferably 7 to 55 ethylene oxide units per molecule and obtainable in a conventional manner by alkoxylation of suitable starter molecules (for example in Ullmanns Encyclopädie der technischen Chemie, 4th edition, volume 19, Verlag Chemie, Weinheim pages 31-38). These are either pure polyethylene oxide ethers or mixed polyalkylene oxide ethers, containing at least 30 mol % and preferably at least 40 mol % of ethylene oxide units, based on all alkylene oxide units present.

Particularly preferred nonionic compounds are monofunctional mixed polyalkylene oxide polyethers having 40 to 100 mol % of ethylene oxide units and 0 to 60 mol % of propylene oxide units.

Useful starter molecules for such nonionic hydrophilicizing agents include saturated monoalcohols such as methanol, ethanol, n-propanol, isopropanol, n-butanol, isobutanol, sec-butanol, the isomers pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or oleic alcohol, aromatic alcohols such as phenol, the isomeric cresol or methoxyphenols, araliphatic alcohols such as benzyl alcohol, anisal alcohol or cinnamyl alcohol, secondary monoamines such as dimethylamine, diethylamine, dipropylamine, diisopropylamine, dibutylamine, bis(2-ethylhexyl)amine, N-methylcyclohexylamine, N-ethylcyclohexylamine or dicyclohexylamine and also heterocyclic secondary amines such as morpholine, pyrrolidine, piperidine or 1H pyrazole. Preferred starter molecules are saturated monoalcohols of the aforementioned kind. Particular preference is given to using diethylene glycol monobutyl ether or n-butanol as starter molecules.

Useful alkylene oxides for the alkoxylation reaction are in particular ethylene oxide and propylene oxide, which can be used in any desired order or else in admixture in the alkoxylation reaction.

Component B1) may utilize organic di- or polyamines such as for example 1,2-ethylene-diamine, 1,2-diaminopropane, 1,3-diaminopropane, 1,3-diaminobutane, 1,6-diaminohexane, isophoronediamine, isomeric mixture of 2,2,4- and 2,4,4-trimethyl-hexamethylenediamine, 2-methylpentamethylenediamine, diethylenetriamine, 4,4-diaminodicyclohexylmethane and/or dimethylethylenediamine.

Component B1) can further utilize compounds which as well as a primary amino group also have secondary amino groups or which as well as an amino group (primary or secondary) also have OH groups. Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

Component B1) can further utilize monofunctional isocyanate-reactive amine compounds, for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide-amines formed from diprimary amines and monocarboxylic acids, monoketimes of diprimary amines, primary/tertiary amines, such as N,N-dimethylaminopropylamine.

Useful anionically hydrophilicizing compounds for component B2) include alkali metal salts of the mono- and diamino sulphonic acids. Examples of such anionic hydrophilicizing agents are salts of 2-(2-aminoethylamino)ethanesulphonic acid, ethylenediaminepropylsulphonic acid, ethylenediaminebutylsulphonic acid, 1,2- or 1,3-propylenediamine-β-ethylsulphonic acid or taurine. It is further possible to use the salt of cyclohexylaminopropanesulphonic acid (CAPS) from WO-A 01/88006 as an anionic hydrophilicizing agent.

Particularly preferred anionic hydrophilicizing agents B2) are those which contain sulphonate groups as ionic groups and two amino groups, such as the salts of 2-(2-aminoethylamino)ethylsulphonic acid and 1,3-propylenediamine-β-ethylsulphonic acid.

Mixtures of anionic and nonionic hydrophilicizing agents can also be used.

A preferred embodiment for producing the specific polyurethane dispersions utilizes components A1) to A4) and B1) to B2) in the following amounts, the individual amounts always adding up to 100% by weight:

5% to 40% by weight of component A1),
55% to 90% by weight of A2),
0.5% to 20% by weight of the sum total of components A3) and B1)
0.1% to 25% by weight of the sum total of the components A4) and B2), with 0.1 to 5% by weight of anionic or potentially anionic hydrophilicizing agents from A4) and/or B2) being used, based on the total amount of components A1) to A4) and B1) to B2).

A particularly preferred embodiment for producing the specific polyurethane dispersions utilizes components A1) to A4) and B1) to B2) in the following amounts, the individual amounts always adding up to 100% by weight:

5% to 35% by weight of component A1),
60% to 90% by weight of A2),
0.5% to 15% by weight of the sum total of components A3) and B1)
0.1% to 15% by weight of the sum total of the components component A4) and B2), with 0.2 to 4% by weight of anionic or potentially anionic hydrophilicizing agents from A4) and/or B2) being used, based on the total amount of components A1) to A4) and B1) to B2).

A very particularly preferred embodiment for producing the specific polyurethane dispersions utilizes components A1) to A4) and B1) to B2) in the following amounts, the individual amounts always adding up to 100% by weight:

10% to 30% by weight of component A1),
65% to 85% by weight of A2),
0.5% to 14% by weight of the sum total of components A3) and B1)
0.1% to 13.5% by weight of the sum total of the components A4) and B2), with 0.5 to 3.0% by weight of anionic or potentially anionic hydrophilicizing agents from A4) and/or B2) being used, based on the total amount of components A1) to A4) and B1) to B2).

The production of the specific polyurethane dispersions can be carried out in one or more stages in homogeneous phase or, in the case of a multistage reaction, partly in disperse phase. After completely or partially conducted polyaddition from A1) to A4) a dispersing, emulsifying or dissolving step is carried out. This is followed if appropriate by a further polyaddition or modification in disperse phase.

Any prior art process can be used, examples being the prepolymer mixing process, the acetone process or the melt dispersing process. The acetone process is preferred.

Production by the acetone process typically involves the constituents A2) to A4) and the polyisocyanate component A1) being to produce an isocyanate-functional polyurethane prepolymer wholly or partly introduced as an initial charge and optionally diluted with a water-miscible but isocyanate-inert solvent and heated to temperatures in the range from 50 to 120° C. The isocyanate addition reaction can be speeded using the catalysts known in polyurethane chemistry.

Useful solvents include the customary aliphatic, keto-functional solvents such as acetone, 2-butanone, which can be added not just at the start of the production process but also later, optionally in portions. Acetone and 2-butanone are preferred and acetone is particularly preferred.

Subsequently, any constituents of A1) to A4) not added at the start of the reaction are added.

In the production of the polyurethane prepolymer from A1) to A4), the amount of substance ratio of isocyanate groups to isocyanate-reactive groups is in the range from 1.05 to 3.5, preferably in the range from 1.1 to 3.0 and more preferably in the range from 1.1 to 2.5.

The reaction of components A1) to A4) to form the prepolymer is effected partially or completely, but preferably completely. Polyurethane prepolymers containing free isocyanate groups are obtained in this way, without a solvent or in solution.

Subsequently, in a further process step, the prepolymer obtained is dissolved with the aid of aliphatic ketones such as acetone or 2-butanone, if this has not been done yet or only to some extent.

In the chain extension of step B), NH₂- and/or NH-functional components are reacted with the still remaining isocyanate groups of the prepolymer. Preferably, the chain extension/termination is carried out before dispersion in water.

Useful chain-extending components include organic di- or polyamines B1) such as for example ethylenediamine, 1,2-diaminopropane, 1,3-diaminopropane, 1,4-diaminobutane, 1,6-diaminohexane, isophoronediamine, isomeric mixture of 2,2,4- and 2,4,4-trimethylhexamethylenediamine, 2-methylpentamethylenediamine, diethylenetriamine, diaminodicyclohexylmethane and/or dimethylethylendiamine.

In addition, it is also possible to employ compounds B1) which, as well as a primary amino group, also have secondary amino groups or which have OH groups as well as an amino group (primary or secondary). Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine for chain extension or termination.

Chain termination is typically carried out using amines B1) having an isocyanate-reactive group such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl-(methyl)aminopropylamine, morpholine, piperidine or suitable substituted derivatives thereof, amide amines formed from diprimary amines and monocarboxylic acids, monoketimes of diprimary amines, primary/tertiary amines, such as N,N-dimethylaminopropylamine.

When chain extension is carried out using anionic hydrophilicizing agents conforming to definition B2) with NH₂ or NH groups, the chain extension of the prepolymers is preferably carried out before dispersion.

The degree of chain extension, i.e. the equivalent ratio of NCO-reactive groups of the compounds used for chain extension and chain termination to free NCO groups of the prepolymer, is between 40 to 150%, preferably between 50 to 120% and more preferably between 60 to 120%.

The aminic components B1) and B2) can optionally be used in water- or solvent-diluted form in the process of the present invention, individually or in mixtures, any order of addition being possible in principle.

When water or organic solvent is used as a diluent, the diluent content of the chain-extending component used in B) is preferably in the range from 70% to 95% by weight.

Dispersion is preferably carried out following chain extension. For dispersion, the dissolved and chain-extended polyurethane polymer is either introduced into the dispersing water, if appropriate by substantial shearing, such as vigorous stirring for example, or conversely the dispersing water is stirred into the chain-extended polyurethane polymer solutions. It is preferable to add the water to the dissolved chain-extended polyurethane polymer.

The solvent still present in the dispersions after the dispersing step is then typically removed by distillation. Removal during the dispersing step is likewise possible.

The residual level of organic solvents in the dispersions which are essential to the present invention is typically less than 1% by weight and preferably less than 0.5% by weight, based on the entire dispersion.

The pH of the dispersions which are essential to the present invention is typically less than 8.0, preferably less than 7.5 and more preferably between 5.5 and 7.5.

As well as the dispersions (I), the compositions to be frothed may also contain auxiliary and additive materials (II).

Examples of such auxiliary and additive materials (II) are foam auxiliaries such as foam formers and stabilizers, thickeners or thixotroping agents, antioxidants, light stabilizers, emulsifiers, plasticizers, pigments, fillers and flow control agents.

Preferably, foam auxiliaries such as foam formers and stabilizers are included as auxiliary and additive materials (II). Useful foam auxiliaries include commercially available compounds such as fatty acid amides, hydrocarbyl sulphates or sulphonates or fatty acid salts, in which case the lipophilic radical preferably contains 12 to 24 carbon atoms and also alkylpolyglycosides obtainable in a conventional manner by reaction of comparatively long-chain monoalcohols (4 to 22 carbon atoms in the alkyl radical) with mono-, di- or polysaccharides (see for example Kirk-Othmer, Encyclopedia of Chemical Technology, John Wiley & Sons, Vol. 24, p. 29).

Preferred foam auxiliaries are sulphosuccinamides, alkanesulphonates or alkyl sulphates having 12 to 22 carbon atoms in the hydrocarbyl radical, alkylbenzenesulphonates or alkylbenzene sulphates having 14 to 24 carbon atoms in the hydrocarbyl radical or fatty acid amides or fatty acid salts having 12 to 24 carbon atoms.

Such fatty acid amides are preferably based on mono- or di-(C2-C3-alkanol)amines. The fatty acid salts may be for example alkali metal salts, amine salts or unsubstituted ammonium salts.

Such fatty acid derivatives are typically based on fatty acids such as lauric acid, myristic acid, palmitic acid, oleic acid, stearic acid, ricinoleic acid, behenic acid or arachidic acid, coco fatty acid, tallow fatty acid, soya fatty acid and their hydrogenation products.

Particularly preferred foam auxiliaries are mixtures of sulphosuccinamides and ammonium stearates, these preferably containing 20% to 60% by weight and more preferably 30% to 50% by weight of ammonium stearates and preferably 80% to 40% by weight and more preferably 70% to 50% by weight of sulphosuccinamides.

Commercially available thickeners can be used, such as derivatives of dextrin, of starch or of cellulose, examples being cellulose ethers or hydroxyethylcellulose, organic wholly synthetic thickeners based on polyacrylic acids, polyvinylpyrrolidones, polymethacrylic compounds or polyurethanes (associative thickeners) and also inorganic thickeners, such as bentonites or silicas.

Frothing in the process of the present invention is accomplished by mechanical stirring of the composition at high speeds of rotation by shaking or by decompressing a blowing gas.

Mechanical frothing can be effected using desired mechanical stirring, mixing and dispersing techniques. Air is generally introduced, but nitrogen and other gases can also be used for this purpose.

The foam thus obtained is, in the course of frothing or immediately thereafter, applied to a substrate or introduced into a mould and dried.

Application to a substrate can be for example by pouring or blade coating, but other conventional techniques are also possible. Multilayered application with intervening drying steps is also possible in principle.

A satisfactory drying rate for the foams is observed at a temperature as low as 20° C., so that drying on injured human or animal tissue presents no problem. However, temperatures above 30° C. are preferably used for more rapid drying and fixing of the foams. However, drying temperatures should not exceed 200° C., preferably 150° C. and more preferably 130° C., since undesirable yellowing of the foams can otherwise occur, inter alia. Drying in two or more stages is also possible.

Drying is generally effected using conventional heating and drying apparatus, such as (circulating air) drying cabinets, hot air or IR radiators.

Application and drying can each be carried out batchwise or continuously, but the entirely continuous process is preferred.

Useful substrates include papers or films which facilitate simple detachment of the wound contact material before it is used to cover an injured site. Human or animal tissue such as skin can similarly serve as a substrate, so that direct closure of an injured site is possible by a wound contact material produced in situ.

The present invention further provides the wound contact materials obtainable by the process of the present invention.

Before drying, the foam densities of the wound contact materials are typically in the range from 50 to 800 g/litre, preferably in the range from 100 to 500 g/litre and more preferably in the range from 100 to 250 g/litre (mass of all input materials [in g] based on the foam volume of one litre).

After drying, the wound contact materials have a microporous, open-cell structure comprising intercommunicating cells. The density of the dried foams is typically below 0.4 g/cm³, preferably below 0.35 g/cm³, more preferably in the range from 0.01 to 0.3 g/cm³ and most preferably in the range from 0.15 to 0.3 g/cm³.

The DIN EN 13726-1 Part 3.2 absorbency with regard to physiological saline is typically 100 and 1500%, preferably in the range from 300 to 1500% and more preferably in the range from 300 to 800% for the polyurethane foams (mass of absorbed liquid based on the mass of dry foam). The DIN EN 13726-2 Part 3.2 water vapour transmission rate is typically in the range from 2000 to 8000 g/24 h*m² and preferably in the range from 3000 to 8000 g/24 h*m², and more preferably in the range from 3000 to 5000 g/24*m².

The polyurethane foams exhibit good mechanical strength and high elasticity. Typically, maximum stress is greater than 0.2 N/mm² and maximum extension greater than 250%. Preferably, maximum stress is greater than 0.4 N/mm² and the extension is greater than 350% (determined according to DIN 53504).

After drying, the thickness of the wound contact materials is typically in the range from 0.1 mm to 50 mm, preferably in the range from 0.5 mm to 20 mm, more preferably in the range from 1 to 10 mm and most preferably in the range from 1 to 5 mm.

The wound contact materials can moreover be adhered, laminated or coated to with further materials, for example materials based on hydrogels, (semi-) permeable films, coatings, hydrocolloids or other foams.

If appropriate, a sterilizing step can be included in the process of the present invention. It is similarly possible in principle for wound contact materials obtainable by the process of the present invention to be sterilized after they have been produced. Conventional sterilizing processes are used where sterilization is effected by thermal treatment, chemical substances such as ethylene oxide or irradiation with gamma rays for example.

It is likewise possible to add, incorporate or coat with antimicrobially or biologically active components which for example have a positive effect with regard to wound healing and the avoidance of germ loads.

In a preferred embodiment of the invention, the active component is selected from the group of the antiseptics, growth factors, protease inhibitors and/or non-steroidal anti-inflammatories/opiates.

In a preferred embodiment of the present invention, the active component comprises an antiseptic biguanide. Biguanides are compounds derived from biguanide (C₂H₇N₅), in particular its polymers. Antiseptic biguanides are biguanides that have an antimicrobial effect, i.e. act as bacteriostats or preferably as bactericides. The compounds in question preferably have a broad effect against many bacteria and can be characterized by a minimal microbicidal concentration (MMC, measured in the suspension test) of at least 0.5 μg/ml, preferably at least 12 or at least 25 μg/ml with regard to E. coli.

A preferred antiseptic biguanide according to this invention is poly(imino[imino-carbonyl]iminopolymethylene), the use of poly(hexamethylene)biguanide (PHMB), also known as polyhexanide, as antiseptic biguanide being particularly preferred. The term “antiseptic biguanides” according to this invention also comprehends metabolites and/or prodrugs of antiseptic biguanides. Antiseptic biguanides can be present as racemates or pure isoforms.

The wound contact materials of the present invention preferably contain antiseptic biguanide and/or its salt, preferably the hydrochloride, in a concentration of 0.01% to 20% by weight, the concentration of 0.1% to 5% by weight being particularly advantageous. The biguanide may have any desired molecular weight distribution.

Owing to the wide utility of the process of the present invention and of the wound contact materials obtainable thereby, it is possible in principle to use said process in the industrial production of wound contact materials. But it is similarly also possible to use it for producing sprayed plasters for example, in which case the wound contact material is formed by direct application of the composition to a wound and simultaneous frothing and subsequent drying.

For industrial production of wound contact materials, the polyurethane dispersion (I) is mixed with foam auxiliaries of the aforementioned kind and thereafter mechanically frothed by introduction of a gas such as air. This foam is applied to a substrate and physically dried. Owing to higher productivity, drying is typically carried out at elevated temperatures in the range from 30 to 200° C., preferably in the range from 50 to 150° C. and more preferably in the range from 60 to 130° C. Preference is further given to an at least two-stage drying beginning at temperatures of 40 to 80° C. and with subsequent further drying at elevated temperatures of 80 to 140° C. Drying is generally carried out using conventional heating and drying apparatuses, for example (circulating air) drying cabinets. Application and drying can each be carried out batchwise or continuously, but preference is given to the wholly continuous process. For sterilization, a sterilizing step can be carried out during or after the process, by irradiation or addition of suitable substances.

When the composition which is essential to the present invention is used to produce a spray plaster, the polyurethane dispersion (I) is formulated with a foam auxiliary and a blowing agent, so that frothing ensues coterminous with spraying. To consolidate the foam formed, the foam is subsequently dried, for which temperatures of 20 to 40° C. are sufficient. When additional heat sources such as a hair dryer or an IR red light lamp are used, however, forced thermal drying up to a maximum temperature of 80° C. is possible.

The blowing agents used are well known from polyurethane chemistry. n-Butane, i-butane and propane and also mixtures thereof are suitable for example, as is also dimethyl ether for example. Preference is given to using a mixture of n-butane, i-butane and propane, whereby the desired, fine-cell foams are obtained. The blowing agent or blowing agent mixture is typically used in an amount of 1% to 50% by weight, preferably 5% to 40% by weight and more preferably 5% to 20% by weight, the sum total of polyurethane dispersion (I) used, blowing agent (mixture) and also optional auxiliary and addition materials (II) being 100% by weight. Spray plasters are preferably provided in spray cans. Pouring of the composition is possible as well as spraying.

### EXAMPLES

Unless indicated otherwise, all percentages are by weight.

Unless indicated otherwise, all analytical measurements relate to temperatures of 23° C.

Solids contents were determined in accordance with DIN-EN ISO 3251.

NCO contents were, unless expressly mentioned otherwise, determined volumetrically in accordance with DIN-EN ISO 11909.

Free NCO groups were monitored by IR spectroscopy (band at 2260 cm¹).

The reported viscosities were determined by rotary viscometry in accordance with DIN 53019 at 23° C. using a rotary viscometer from Anton Paar Germany GmbH, Ostfildern, Germany.

### Substances and Abbreviations Used:

Diaminosulphonate: NH₂—CH₂CH₂—NH—CH₂CH₂—SO₃Na (45% in water)
Desmophen® 2020/C2200: polycarbonate polyol, OH number 56 mg KOH/g, number average molecular weight 2000 g/mol (Bayer MaterialScience AG, Leverkusen, Germany)
PolyTHF® 2000: polytetramethylene glycol polyol, OH number 56 mg KOH/g, number average molecular weight 2000 g/mol (BASF AG, Ludwigshafen, Germany)
PolyTHF® 1000: polytetramethylene glycol, OH number 112 mg KOH/g, number average molecular weight 1000 g/mol (BASF AG, Ludwigshafen, Germany)
LB 25 polyether: monofunctional polyether based on ethylene oxide/propylene oxide, number average molecular weight 2250 g/mol, OH number 25 mg KOH/g (Bayer Material Science AG, Leverkusen, Germany)
Stokal® STA: foam auxiliary based on ammonium stearate, active content: 30% (Bozzetto GmbH, Krefeld, Germany)
Stokal® SR: foam auxiliary based on succinamate, active content: about 34% (Bozzetto GmbH, Krefeld, Germany)
Simulsol® SL 26: alkylpolyglycoside based on dodecyl alcohol, about 52% strength in water, Seppic GmbH, Cologne, Germany

The determination of the average particle sizes (the number average is reported) of the polyurethane dispersions was carried out using laser correlation spectroscopy (instrument: Malver Zetasizer 1000, Malver Inst. Limited).

### Example 1

### Polyurethane Dispersion 1

987.0 g of PolyTHF® 2000, 375.4 g of PolyTHF® 1000, 761.3 g of Desmophen® C2200 and 44.3 g of LB 25 polyether were heated to 70° C. in a standard stirring apparatus. Then, a mixture of 237.0 g of hexamethylene diisocyanate and 313.2 g of isophorone diisocyanate was added at 70° C. in the course of 5 min and the mixture was stirred at 120° C. until the theoretical NCO value was reached. The ready-produced prepolymer was dissolved with 4830 g of acetone and, in the process, cooled down to 50° C. and subsequently admixed with a solution of 25.1 g of ethylenediamine, 116.5 g of isophoronediamine, 61.7 g of diaminosulphonate and 1030 g of water metered in over 10 min. The mixture was subsequently stirred for 10 min. Then, a dispersion was formed by addition of 1250 g of water. This was followed by removal of the solvent by distillation under reduced pressure.

The white dispersion obtained had the following properties:

Solids content: 61%
Particle size (LKS): 312 nm
Viscosity (viscometer, 23° C.): 241 mPas

### pH (23° C.): 6.02

### Example 2

### Polyurethane Dispersion 2

34.18 g of PolyTHF® 2000, 85.1 g of PolyTHF® 1000, 172.6 g of Desmophen® C2200 and 10.0 g of LB 25 polyether were heated to 70° C. in a standard stirring apparatus. Then, a mixture of 53.7 g of hexamethylene diisocyanate and 71.0 g of isophorone diisocyanate was added at 70° C. in the course of 5 min and the mixture was stirred at 120° C. until the theoretical NCO value was reached. The ready-produced prepolymer was dissolved with 1005 g of acetone and, in the process, cooled down to 50° C. and subsequently admixed with a solution of 5.70 g of ethylenediamine, 26.4 g of isophoronediamine, 9.18 g of diaminosulphonate and 249.2 g of water metered in over 10 min. The mixture was subsequently stirred for 10 min. Then, a dispersion was formed by addition of 216 g of water. This was followed by removal of the solvent by distillation under reduced pressure.

The white dispersion obtained had the following properties:

Solids content: 63%
Particle size (LKS): 495 nm
Viscosity (viscometer, 23° C.): 133 mPas

### pH (23° C.): 6.92

### Example 3

### Polyurethane Dispersion 3

987.0 g of PolyTHF® 2000, 375.4 g of PolyTHF® 1000, 761.3 g of Desmophen® C2200 and 44.3 g of LB 25 polyether were heated to 70° C. in a standard stirring apparatus. Then, a mixture of 237.0 g of hexamethylene diisocyanate and 313.2 g of isophorone diisocyanate was added at 70° C. in the course of 5 min and the mixture was stirred at 120° C. until the theoretical NCO value was reached. The ready-produced prepolymer was dissolved with 4830 g of acetone and, in the process, cooled down to 50° C. and subsequently admixed with a solution of 36.9 g of 1,4-diaminobutane, 116.5 g of isophoronediamine, 61.7 g of diaminosulphonate and 1076 g of water metered in over 10 min. The mixture was subsequently stirred for 10 min. Then, a dispersion was formed by addition of 1210 g of water. This was followed by removal of the solvent by distillation under reduced pressure.

The white dispersion obtained had the following properties:

Solids content: 59%
Particle size (LKS): 350 nm
Viscosity (viscometer, 23° C.): 126 mPas

### pH (23° C.): 7.07

### Example 4

### Polyurethane Dispersion 4

201.3 g of PolyTHF® 2000, 76.6 g of PolyTHF® 1000, 155.3 g of Desmophen® C2200, 2.50 g of 1,4-butanediol and 10.0 g of LB 25 polyether were heated to 70° C. in a standard stirring apparatus. Then, a mixture of 53.7 g of hexamethylene diisocyanate and 71.0 g of isophorone diisocyanate was added at 70° C. in the course of 5 min and the mixture was stirred at 120° C. until the theoretical NCO value was reached. The ready-produced prepolymer was dissolved with 1010 g of acetone and, in the process, cooled down to 50° C. and subsequently admixed with a solution of 5.70 g of ethylenediamine, 26.4 g of isophoronediamine, 14.0 g of diaminosulphonate and 250 g of water metered in over 10 min. The mixture was subsequently stirred for 10 min. Then, a dispersion was formed by addition of 243 g of water. This was followed by removal of the solvent by distillation under reduced pressure.

The white dispersion obtained had the following properties:

Solids content: 62%
Particle size (LKS): 566 nm
Viscosity (viscometer, 23° C.): 57 mPas

### pH (23° C.): 6.64

### Example 5

### Polyurethane Dispersion 5

201.3 g of PolyTHF® 2000, 76.6 g of PolyTHF® 1000, 155.3 g of Desmophen® C2200, 2.50 g of trimethylolpropane and 10.0 g of LB 25 polyether were heated to 70° C. in a standard stirring apparatus. Then, a mixture of 53.7 g of hexamethylene diisocyanate and 71.0 g of isophorone diisocyanate was added at 70° C. in the course of 5 min and the mixture was stirred at 120° C. until the theoretical NCO value was reached. The ready-produced prepolymer was dissolved with 1010 g of acetone and, in the process, cooled down to 50° C. and subsequently admixed with a solution of 5.70 g of ethylenediamine, 26.4 g of isophoronediamine, 14.0 g of diaminosulphonate and 250 g of water metered in over 10 min. The mixture was subsequently stirred for 10 min. Then, a dispersion was formed by addition of 293 g of water. This was followed by removal of the solvent by distillation under reduced pressure.

The white dispersion obtained had the following properties:

Solids content: 56%
Particle size (LKS): 440 nm
Viscosity (viscometer, 23° C.): 84 mPas

### pH (23° C.): 6.91

### Example 6

### Polyurethane Dispersion 6

1072 g of PolyTHF® 2000, 407.64 g of PolyTHF® 1000, 827 g of Desmophen® C2200 and 48.1 g of LB 25 polyether were heated to 70° C. in a standard stirring apparatus. Then, a mixture of 257.4 g of hexamethylene diisocyanate and 340 g of isophorone diisocyanate was added at 70° C. in the course of 5 min and the mixture was stirred at 120° C. until the theoretical NCO value was reached. The ready-produced prepolymer was dissolved with 4820 g of acetone and, in the process, cooled down to 50° C. and subsequently admixed with a solution of 27.3 g of ethylenediamine, 126.5 g of isophoronediamine, 67.0 g of diaminosulphonate and 1090 g of water metered in over 10 min. The mixture was subsequently stirred for 10 min. Then, a dispersion was formed by addition of 1180 g of water. This was followed by removal of the solvent by distillation under reduced pressure.

The white dispersion obtained had the following properties:

Solids content: 60%
Particle size (LKS): 312 nm
Viscosity (viscometer, 23° C.): 286 mPas

### pH (23° C.): 7.15

### Example 7

54 g of a polyurethane dispersion produced according to Example 2 were mixed with 1.37 g of Simulsol® SL 26 and admixed with 6 g of a blowing agent mixture of 1-butane/propane/n-butane in a suitable aerosol can. Spraying (about 1 cm wet film thickness) and drying C10 min at 120° C. gave a clean white, fine-cell foam.

### Example 8

54 g of a polyurethane dispersion produced according to Example 2 were mixed with 1.37 g of Simulsol® SL 26, and admixed with 6 g of dimethyl ether in a suitable aerosol can. Spraying (about 1 cm wet film thickness) and drying C10 min at 120° C.) gave a clean white, fine-cell foam.

### Comparative Example 1

### Polyurethane Dispersion, not Inventive

### No Sulphonate Groups, Just Hydrophilicization Through Nonionic Groups and Carboxylate Groups

Example 1 is repeated except that the diaminosulphonate was replaced by an equimolar amount of a carboxylato-containing component:

206.8 g of PolyTHF® 2000, 78.7 g of PolyTHF® 1000, 159.5 g of Desmophen® C2200 and 9.3 g of LB 25 polyether were heated to 70° C. in a standard stirring apparatus. Then, a mixture of 49.7 g of hexamethylene diisocyanate and 65.6 g of isophorone diisocyanate was added at 70° C. in the course of 5 min and the mixture was stirred at 120° C. until the theoretical NCO value was reached. The ready-produced prepolymer was dissolved with 1010 g of acetone and, in the process, cooled down to 50° C. and subsequently admixed with a solution of 5.3 g of ethylenediamine, 24.4 g of isophoronediamine, 11.9 g of KV 1386 (40% aqueous solution of the sodium salt of N-(2-aminoethyl)-β-alanine, BASF AG, Ludwigshafen, Germany) and 204 g of water metered in over 10 min. The mixture was subsequently stirred for 10 min. Then, a dispersion was formed by addition of 235 g of water. This was followed by removal of the solvent by distillation under reduced pressure. A total of 250 g of water had to be added because of the high viscosity.

The white dispersion obtained had the following properties:

Solids content: 47%
Particle size (LKS): 918 nm
Viscosity (viscometer, 23° C.): 162 mPas

### pH (23° C.): 7.22

Owing to the comparatively high average particle size of >900 nm and contrary to the purely sulphonate-hydrophilicized dispersions, sedimentation was observed to ensue within a few days, making further processing into wound contact materials difficult.

### Comparative Example 2

### Polyurethane Dispersion, not Inventive

### No Sulphonate Groups, Just Hydrophilicization Through Nonionic Groups and Carboxylate Groups

Comparative Example 1 was repeated except that the amount of the carboxylato-containing hydrophilicizing component was increased by 50% (while keeping the degree of chain extension the same).

206.8 g of PolyTHF® 2000, 78.7 g of PolyTHF® 1000, 159.5 g of Desmophen® C2200 and 9.3 g of LB 25 polyether were heated to 70° C. in a standard stirring apparatus. Then, a mixture of 49.7 g of hexamethylene diisocyanate in 65.6 g of isophorone diisocyanate was added at 70° C. in the course of 5 min and the mixture was stirred at 120° C. until the theoretical NCO value was reached. The ready-produced prepolymer was dissolved with 1010 g of acetone and, in the process, cooled down to 50° C. and subsequently admixed with a solution of 5.3 g of ethylenediamine, 21.8 g of isophoronediamine, 17.9 g of KV 1386 (40% aqueous solution of the sodium salt of N-(2-aminoethyl)-β-alanine, BASF AG, Ludwigshafen, Germany) and 204 g of water metered in over 10 min. The mixture was subsequently stirred for 10 min. Then, a dispersion was formed by addition of 235 g of water. This was followed by removal of the solvent by distillation under reduced pressure.

The white dispersion obtained had the following properties:

Solids content: 52.2%
Particle size (LKS): 255 nm
Viscosity (viscometer, 23° C.): 176 mPas

### pH (23° C.): 8.31

This polyurethane dispersion had a lower average particle size but a somewhat higher pH than Example 7. Further processing to wound contact materials was distinctly more difficult than with purely sulphonate-hydrophilicized dispersions.

### Examples 9 to 14

### Foams Produced from the Polyurethane Dispersions of Examples 1 to 6

The Table 1 amounts of the polyurethane dispersions produced as described in Examples 1 to 6 were mixed with foam auxiliaries indicated in Table 1 and frothed by means of a commercially available hand stirrer (stirrer made of bent wire) to a 1 litre foam volume. Thereafter, the polyurethane foams were drawn down on silicone-coated paper by means of a blade coater set to a gap height of 4 mm. Table 1 similarly recites the drying conditions for the polyurethane foams produced as indicated. Clean white polyurethane foams having good mechanical properties and fine pore structure were obtained without exception.

TABLE 1

| | Amount [g] | | | |
|---|---|---|---|---|
| | Polyurethane | | | |
| Foam | dispersion | Stokal ® | Stokal ® | |
| No. | (Example) | STA | SR | Curing |
| 9a | 235.0 (1) | 4.2 | 5.6 | 2 h/37° C. |
| 9b | 235.0 (1) | 4.2 | 5.6 | 2 h/37° C., 30 min/110° C. |
| 10 | 235.0 (2) | 4.2 | 5.6 | 2 h/37° C., 30 min/120° C. |
| 11a | 235.0 (3) | 4.2 | 5.6 | 2 h/37° C. |
| 11b | 235.0 (3) | 4.2 | 5.6 | 2 h/37° C., 30 min/120° C. |
| 12a | 235.0 (4) | 4.2 | 5.6 | 2 h/37° C. |
| 12b | 235.0 (4) | 4.2 | 5.6 | 2 h/37° C., 30 min/120° C. |
| 13a | 235.0 (5) | 4.2 | 5.6 | 2 h/37° C. |
| 13b | 235.0 (5) | 4.2 | 5.6 | 2 h/37° C., 30 min/120° C. |
| 14 | 235.0 (6) | 4.2 | 5.6 | 2 h/37° C., 30 min/120° C. |

As is discernible from Table 2, all the polyurethane foams exhibited a very rapid imbibition of water, a high absorption of physiological saline (“free swell absorption”), a very high moisture vapour transmission rate (MVTR) and also good mechanical strength, in particular after moist storage.

TABLE 2

| | | Free | |
|---|---|---|---|
| Foam | Imbibition | absorbency²⁾ | MVTR³⁾ |
| No. | rate¹⁾ [s] | [g/10 cm²] | [g/m² * 24 h] |
| 9a | not | 23.1 | 4300 |
| | determined | | |
| 9b | not | 19.2 | 5000 |
| | determined | | |
| 10 | 3 | 28.4 | 4700 |
| 11a | 4 | 20.6 | 4300 |
| 11b | 14 | 18.3 | 4300 |
| 12a | 4 | 24.7 | 4800 |
| 12b | 7 | 26.7 | 4500 |
| 13a | 5 | 25.5 | 4800 |
| 13b | 7 | 23.1 | 4100 |
| 14 | 4 | 21.3 | not |
| | | | determined |

¹⁾time for complete penetration of a drop (of distilled water) into the foam;
²⁾absorption of physiological saline determined according to DIN EN 13726-1 Part 3.2 (5 instead of 9 test samples);
³⁾moisture vapour transmission rate determined according to DIN EN 13726-2 Part 3.2

### Example 15

Example 15 describes the production of biomedical foam articles containing antiseptic biguanide and particularly PHMB.

Free absorbency was determined by absorption of physiological saline to DIN EN 13726-1 Part 3.2. Moisture vapour transition rate (MVTR) was determined according to DIN EN 13726-2 Part 3.2.

### Example 15.1

### Production of Polyurethane Dispersion 1

1077.2 g of PolyTHF® 2000, 409.7 g of PolyTHF® 1000, 830.9 g of Desmophen® C2200 and 48.3 g of LB 25 polyether were heated to 70° C. in a standard stirring apparatus. Then, a mixture of 258.7 g of hexamethylene diisocyanate and 341.9 g of isophorone diisocyanate was added at 70° C. in the course of 5 min and the mixture was stirred at 120° C. until the theoretical NCO value was reached or the actual NCO value was slightly below the theoretical NCO value. The ready-produced prepolymer was dissolved with 4840 g of acetone and, in the process, cooled down to 50° C. and subsequently admixed with a solution of 27.4 g of ethylenediamine, 127.1 g of isophoronediamine, 67.3 g of diaminosulphonate and 1200 g of water metered in over 10 min. The mixture was subsequently stirred for 10 min. Then, a dispersion was formed by addition of 654 g of water. This was followed by removal of the solvent by distillation under reduced pressure.

The polyurethane dispersion obtained had the following properties:

Solids content: 61.6%
Particle size (LKS): 528 nm

### pH (23° C.): 7.5

### Example 15.2

### Production of Wound Contact Materials/Foams from Polyurethane Dispersion 1

120 g of a polyurethane dispersion produced according to Example 15.1 were mixed with 1.48 g of Plantacare® 1200 UP (previously adjusted to pH 7 with citric acid) and 0.24 g of Stokal® STA and also with 76 mg of polyhexamethylenebiguanide. After 20 minutes' beating and drying (20 min at 120° C.) a clean white, fine-cell, hydrophilic foam was obtained.

### Example 15.3

### Production of Wound Contact Materials/Foams from Polyurethane Dispersion 1

120 g of a polyurethane dispersion produced according to Example 15.1 were mixed with 1.48 g of Plantacare® 1200 UP (previously adjusted to pH 7 with citric acid) and 0.24 g of Stokal® STA and also with 151 mg of polyhexamethylenebiguanide. After 20 minutes' beating and drying (20 min at 120° C.) a clean white, fine-cell, hydrophilic foam was obtained.

### Example 15.4

### Production of Wound Contact Materials/Foams from Polyurethane Dispersion 1

120 g of a polyurethane dispersion produced according to Example 15.1 were mixed with 3.78 g of Pluronic® PE 6800 and also with 76 mg of polyhexamethylenebiguanide. After 20 minutes' beating and drying (20 min at 120° C.) a clean white, fine-cell, hydrophilic foam was obtained.

### Example 15.5

### Production of Wound Contact Materials/Foams from Polyurethane Dispersion 1

120 g of a polyurethane dispersion produced according to Example 15.1 were mixed with 13.40 g of Pluronic® PE 6800 and also with 400 mg of polyhexamethylenebiguanide. After 20 minutes' beating and drying (20 min at 120° C.) a clean white, fine-cell, hydrophilic foam was obtained.

1. A process for producing wound contact materials which comprises compositions containing anionically hydrophilicized, aqueous polyurethane dispersions (I) being frothed and physically dried without chemical crosslinking.

2. A process according to claim 1, wherein the polyurethane dispersions (I) are anionically hydrophilicized by sulphonate groups only.

3. A process according to claim 2, wherein the sulphonate groups have alkali metal cations as counter-ions.

4. A process according to claim 1, wherein the polyurethane dispersions (I) comprise 0.1 to 15 milliequivalents per 100 g of solid resin of anionic or potentially anionic groups based on solid resin.

5. A process according to claim 1, wherein the dispersions (I) have solids contents in the range from 55% to 65% by weight based on the polyurethane present therein.

6. A process according to claim 1, wherein the dispersions (I) are obtainable by
A) isocyanate-functional prepolymers being produced from
A1) organic polyisocyanates
A2) polymeric polyols having number-average molecular weights in the range from 400 to 8000 g/mol and OH functionalities in the range from 1.5 to 6 and
A3) optionally hydroxyl-functional compounds having molecular weights in the range from 62 to 399 g/mol and
A4) optionally isocyanate-reactive, anionic or potentially anionic and optionally nonionic hydrophilicizing agents
and
B) its free NCO groups then being wholly or partly reacted
B1) optionally with amino-functional compounds having molecular weights in the range from 32 to 400 g/mol and
B2) with amino-functional, anionic or potentially anionic hydrophilicizing agents
by chain extension, and the prepolymers being dispersed in water before, during or after step B).

7. A process according to claim 1, wherein the compositions to be frothed contain, as auxiliary and additive materials (II), fatty acid amides, sulphosuccinamides, hydrocarbyl sulphonates or sulphates, alkylpolyglycosides and/or fatty acid salts as foam formers and stabilizers.

8. A process according to claim 7, wherein the mixtures of sulphosuccinamides and ammonium stearates are used as foam formers and stabilizers, these mixtures containing 70% to 50% by weight of sulphosuccinamides.

9. Wound contact materials obtainable by a process according to claim 1.

10. Wound contact materials according to claim 9, wherein they have a microporous, open-cell structure and a density of below 0.4 g/cm³ in the dried state.

11. Wound contact materials according to claim 9, wherein they have a DIN EN 13726-1 Part 3.2 physiological saline absorbency of 100 and 1500% (mass of liquid taken up, based on the mass of dry foam) and a DIN EN 13726-2 Part 3.2 water vapour transmission rate in the range from 2000 to 8000 g/24 h*m².

12. Wound contact materials according to claim 9, wherein they also contain an active component selected from the group consisting of antiseptics, growth factors, protease inhibitors and non-steroidal anti-inflammatories/opiates.

13. Wound contact materials according to claim 12, wherein the active component contains an antiseptic biguanide.

14. Wound contact material according to claim 13, wherein the antiseptic biguanide is poly(hexamethylene)biguanide (PHMB).
